(19) United States
(12) Patent Application Publication
(10) Pub. No.: US 2007/0173752 A1
(43) Pub. Date: Jul. 26, 2007

(54) Integrated package

(76) Inventor: Troels Schonfeldt, Copenhagen NV (DK)

Correspondence Address: JACOBSON HOLMAN PLLC, 400 SEVENTH STREET N.W., SUITE 600, WASHINGTON, DC, 20004, US

(21) Appl. No.: 10/581,049

(22) PCT Filed: Nov. 26, 2004
(86) PCT No.: PCT/DK04/00826

§ 371 Date: Apr. 2, 2007

Related U.S. Application Data
(60) Provisional application No. 60/528,190, filed on Dec. 10, 2003.

(30) Foreign Application Priority Data
Nov. 28, 2003 (DK) PA 2003 01763
Aug. 31, 2004 (DK) PA 2004 01319

(51) Int. Cl. A61F 15/00; A61F 13/00

(52) U.S. Cl. 602/57; 602/41

(57) ABSTRACT

A layered product for non-touch application of an adhesive article comprising a cover layer inseparably attached to an adhesive layer, said adhesive article being interposed between a top layer and a bottom layer where the top layer is situated proximate to the cover layer and the bottom layer situated proximate to the adhesive layer, said bottom layer having a release layer which is releasable attached to a first end portion of the adhesive surface of said adhesive layer and where a second end portion of the adhesive surface of said adhesive layer is releasable attached to a first portion of a tap member which is attached to the top layer, or formed from a part of the top layer, and a second portion of said tab member forming a layer situated on the top layer.

The present invention related to an adhesive article integrated in a package, wherein the layers enclosing the adhesive article may be used, not only to isolate the adhesive article from its surroundings, but also as part of a grip for opening of the package and as a dimensionally stable backing layer for applying the adhesive article to a surface, such as a skin surface, using only one hand.

### BACKGROUND OF THE INVENTION

It is desirable to be able to apply an adhesive article such as a wound dressing without touching the adhesive layer in order to avoid reduction of the adhesiveness of the article. Also, it is desirable to be able to apply a sterile adhesive wound dressing to a patient's skin without touching the surface of the dressing in order to avoid transferring bacteria into the wound.

Furthermore, it is desirable that the adhesive product may be applied to the skin using only one hand, in particular if the patient has to place the adhesive product on a place of the body where only one of the patient's hands is available.

The prior art discloses several methods for facilitating handling of wound dressings and one method is described in U.S. Pat. No. 5,106,629. The product of this patent is constituted of three layers: a dimensionally stable backing layer, an adhesive layer and a release layer. When applying the dressing the release layer is removed by using an extending tab attached thereto, to expose the adhesive layer. The remaining layers of the wound dressing are then applied to the wound site with the adhesive layer directly contacting the wound. Once these layers are in place, the dimensionally stable backing member is removed, preferably using an extending tab attached hereto.

The object of the invention described in WO 97/43991 is to ensure easy handling of a wound dressing. The wound dressing comprises a main part and a handle part, the main part comprising a carrier layer, an adhesive layer and a release layer and the handle part comprising one or more tab members designed for use as a “non-touch” grip when applying the dressing to the skin. The tab member and the main part of the dressing have at least one layer in common, but do not have all layers in common, leading to a reduction in the force needed to remove the tab member after applying the dressing.

These two documents suggest how to handle a wound dressing without touching the adhesive layer during application, but they do not combine packaging of the products with non-touch application of the products. Furthermore, as the release layer is initially removed for exposing the adhesive surface and as the release layer is not kept attached to the other parts that have to be thrown out, the user will have an extra part to collect and throw out after the adhesive article has been applied.

U.S. Pat. No. 5,840,052 also describes an adhesive dressing (cover layer and adhesive layer) wherein the cover layer is releasable attached to a support layer which has a portion extending beyond the cover layer and is folded thereby covering a part of the adhesive layer and folded back upon itself to form a gripping portion for removal of the support layer after the adhesive dressing has been applied to the skin. The portion of the adhesive layer not covered by the support layer is releasable attached to a release layer, which is removed before application of the adhesive dressing to the skin. To be sterile or isolated from the environment the product of this patent has to be placed in a separate closed package. The documents does not teach how to isolate the adhesive article from the environment. This product also has the problem of extra parts that will have to be collected and thrown out after application of the adhesive article

A third product is described in WO 98/00080. This medical adhesive composite, e.g. a dressing, is combined with a package. The packaged product comprises a top sheet of packaging material, a carrier material, a backing material, a pressure sensitive adhesive and a bottom sheet of packaging material with a release surface. The carrier material is preferably substantially more rigid than the backing material in order to prevent the backing from wrinkling or folding onto itself in whole or in part during application of the dressing. The carrier material is capable of being attached to the backing by any suitable method, such as heat sealing, adhesives, mechanical bonds, wax coatings etc. The bond is secure, yet releasable, i.e. the carrier and backing can be separated without destroying the integrity of the backing or the bond between the adhesive on the backing and the skin of a patient. In addition the bond between the carrier and the backing should be stronger than the bond between the adhesive on the bottom face of the backing and the release liner or surface of the packaging. Adhering the medical adhesive composites directly to the bottom sheet of the packaging material rather than including a separate release liner on the product simplifies the process of dispensing the medical adhesive composites. The bond strength between the release surface and the bottom sheet is greater than the bond strength between the release surface and the adhesive on the bottom face of the backing.

This document demonstrates how it is possible to include packaging of the product into a single process but the process used is complicated and the packaged product may be difficult to apply.

U.S. Pat. No. 5,511,689 discloses a paper with several discrete plasters or adhesive-backed articles, which enable the product to be kept sterile until in use. The disclosed product comprises an adhesive with a cover layer, which is releasable attached to the top layer by an adhesive present on the top layer, which is also used to adhere the top layer of to the bottom layer thereby enclosing the adhesive-backed article. The product disclosed in this document is therefore in the form of a sealed package and it is suitable for non-touch application using only one hand. This solution requires that the bond between the cover layer and the top layer is weaker than the bond between the adhesive article and the skin.

WO 00/30580 relates to a layered product, such as an adhesive dressing, for non-touch application comprising an adhesive dressing (adhesive layer and cover layer), a top layer and a bottom layer releasable sealed to each other to form a package containing the adhesive dressing, wherein the cover layer is releasable attached to the top layer and the adhesive layer is attached to a release layer. When used the bottom layer of the package is removed together with the release layer while the top layer stays attached to the cover layer and is used to place the adhesive dressing correctly to the skin. It is not described how the carrier layer is releasable attached to the top layer.

A drawback to these products is that careful control of the bond strength of the bond between the layers are necessary in order to achieve a product which may be applied to the skin without deformation and wrinkling of the adhesive product.

U.S. Pat. No. 5,099,832 discloses an adhesive dressing (cover layer and adhesive layer) in a package wherein the adhesive surface is releasable attached to a folded release strip in one end and another strip in the other end. The cover layer of the adhesive dressing is reversibly attached to a support. The adhesive dressing and the package are two separate units and the adhesive dressing has to be pulled out of the package before the release strips may be removed and the adhesive dressing applied to the skin. Compared to the integrated package according to the invention, use of this product involves collecting and throwing out several additional pieces of the product after the adhesive article has been applied.

U.S. Pat. No. 5.397,297 discloses an adhesive bandage in a closed package wherein the adhesive surface is releasable attached to a folded release strip which makes it possible to pull out the adhesive bandage from the package while simultaneously removing the release strip exposing most of the adhesive surface. A small part of the package and a release surface still covers one end of the adhesive bandage enabling non-touch application of the adhesive bandage.

However, the lack of a stiff support without elastic properties on the back of the adhesive bandage makes it difficult to apply the bandage to the skin, and even if the adhesive bandage is successfully applied to the skin it may easily become stretched typically leading to removal of the adhesive bandage from the skin and reapplication thereof until the adhesive is placed comfortably on the skin. Furthermore, use of this product involves collecting and throwing out a number of additional pieces of the product compared to the integrated package of the invention. Reapplication of the adhesive article is also known to weaken the adhesiveness of the adhesive article considerably and increase the risk of introducing bacterial into the wound.

None of the above-mentioned documents describes a layered product in the form of an adhesive article integrated in a package with a tab member according to the present invention. Further, with the construction of the package according to the invention, the cover layer need not be attached to the top layer because a small part of the adhesive layer is attached to the tab member, which is attached to the top layer.

According to the invention, separation of the top layer from the cover layer is easily accomplished once the adhesive dressing has been applied to the surface for which it was intended.

The present invention only involves throwing out of one piece of the product, which the user already has in his hand once the adhesive article has been applied.

In addition, the tab member is useful as part of a grip for opening of the package, simultaneously exposing the adhesive surface of the dressing, and placing the adhesive article on the skin using the top layer as an dimensionally stable backing layer. This last aspect is particularly important when adhesive article with a small area or very thin adhesive articles has to be applied to a surface.

### SUMMARY OF THE INVENTION

The object of the invention is to provide a layered product, in particular an integrated package containing a sterile adhesive article, such as a wound dressing, which may be applied to a surface, suitable the skin or a wound in a manner avoiding contact with the adhesive surface and at the same time avoiding stretching and wrinkling of the adhesive article. A further advantage is that the adhesive article may be applied using only one hand.

Thus the present invention relates to a layered product for non-touch application of an adhesive article comprising a cover layer inseparably attached to an adhesive layer, said adhesive article being interposed between a top layer and a bottom layer where the top layer is situated proximate to the cover layer and the bottom layer is situated proximate to the adhesive layer, said bottom layer having a release layer which is releasable attached to a first end portion of the adhesive surface of said adhesive layer and where a second end portion of the adhesive surface of said adhesive layer is releasable attached to a first portion of a tap member which is attached to the top layer, or formed from a part of the top layer, and where a second portion of said tab member forms a layer situated on, or proximate to the top layer. Suitably, the tab member forms a layer situated between the top and bottom layer, partly overlapping with the second portion of the adhesive surface of the adhesive article.

The invention also relates to a method for application of the adhesive article of the layered product of the invention to the surface of a living being.

### BRIEF DESCRIPTION OF THE DRAWINGS

The invention is disclosed in more detail with reference to the drawings, in which:

FIG. 1 is a longitudinal cross-sectional view of the layered product of the invention and its essentially flat, planar layers.

FIG. 2 is essentially the same figure as FIG. 1, but illustrates the embodiment of the invention where the top and bottom layer are sealed to each other forming a closed package.

FIG. 3 illustrates the layered product of the invention after the top layer 2 and the bottom layer 1 has been pulled apart and the adhesive surface of the adhesive layer 4 has been exposed.

FIG. 4 also illustrates the layered product of the invention after the top layer 2 and the bottom layer 1 have been pulled apart and the adhesive surface of the adhesive layer 4 has been exposed, but from another angle.

FIG. 5 illustrates how the top and bottom layers may be pulled apart and the adhesive layer inseparably attached to the cover layer may be applied to the skin using only one hand and using the top layer as a support during application of the adhesive article to the skin.

FIG. 6 illustrates different shapes of the tab member.

FIG. 7 and 8 illustrates the positioning of release layers and welding portions in a layered product of the invention.

### DETAILED DESCRIPTION OF THE PRESENT INVENTION

As used herein, the terms “fastened to” or “attached to” means “releasable attached to” or “inseparably attached to”.

As used herein “releasable attached to”, means that the two layers may be separated without permanently deforming or damaging the layers involved.

As used herein, the expression “inseparably attached to” means that the layers cannot be separated while keeping both layers intact.

According to the invention, the top layer and bottom layer may be and are preferably fastened to each other at least at their edges opposite to the position of the tab member, suitably by welding or by the use of an adhesive. According to a preferred embodiment of the invention, the top layer and bottom layer are made from separate sheets, which are inseparably attached to each other, suitably by welding, at their edges opposite the position of the tab member in the opposite end of the top layer.

According to another embodiment of the invention, the top layer and bottom layer are made from one sheet folded over upon itself to form an edge in the position opposite to the position of the tab member on the top layer.

According to a further embodiment of the invention, both the bottom layer and the top layer have a surface area that is larger than the area of the adhesive article.

According to this embodiment of the invention, where the bottom layer and the top layer have a surface area larger than the area of the adhesive article, and the top and bottom layer and the top layer and tab member may be releasable sealed to each thereby form a sealed package containing the adhesive article. According to this embodiment of the invention, a sterile adhesive article in a sterile package may be provided.

As used herein the word “sealed” means that the layers are releasable and/or inseparably attached to each other and forms a seal isolating the adhesive article from the environment. Releasable sealed, means that the seal may be broken and the layers may be separated without damaging the layers. It does not mean that the bottom and top layer cannot be fastened or attached (including inseparably attached) to each other, for example at the edge opposite the position of the tap member.

The sealing around the adhesive article may be provided by any suitable means including heat sealing, contact adhesives, cohesive materials, mechanical bonds, etc. Regardless of the sealing mechanism, it is preferred that it is compatible with the sterilization process used, e.g. gamma irradiation, ethylene oxide, etc.

In one embodiment of the invention, an area of the bottom layer situated peripherally to the release layer on the bottom layer is covered by an adhesive layer, which is releasable attached to the top layer, suitable the bottom layer is releasable sealed to the top layer.

According to a second embodiment of the invention, an area of the top layer situated peripherally to the cover layer of the adhesive article, and optionally a part of the tab member, is covered by an adhesive layer, which is releasable attached to the a peripheral portion of the bottom layer, suitable the top layer is releasable sealed to the bottom layer.

According to a third embodiment of the invention, an area of the bottom layer situated peripherally to the release layer on the bottom layer is covered by a cohesive layer and the surface of the top layer and tab member facing the bottom layer is covered by a cohesive layer essentially forming a mirror image of the cohesive layer on the bottom surface, said cohesive layers releasable attaching the top layer to the bottom layer. Preferably, the top and bottom layers are releasable sealed to each other by the cohesive layers.

According to the invention, a first end portion of the adhesive surface of the adhesive article is releasable attached to a release layer on the bottom layer and a second end portion of the adhesive surface of the adhesive article is releasable attached to the tab member.

In this context, the term “end portion” means a smaller or lager part of the adhesive surface at one end of the adhesive layer. Thus, an end portion may comprise more than 50% of the adhesive surface of the adhesive layer or it may comprise less than 50% of the adhesive surface of the adhesive layer.

Suitably, more than 50% of the end portion of the adhesive surface of the adhesive article is releasable attached to the release layer situated on the bottom layer and less than 50% of an end portion of the adhesive surface of the adhesive article is releasable attached to the tab member, more suitably more than 85% of the end portion of the adhesive surface of the adhesive article is releasable attached to the release layer situated on the bottom layer and less than 15% of the other end portion of the adhesive surface of the adhesive article is releasable attached to the tab member.

According to one embodiment of the invention, the tab member is a separate layer fastened to the top layer. Suitably, the tab member is fastened to the top layer at an edge of the top layer or proximate to an edge of the top layer. One method for attaching the tab member to the top layer is by welding.

In one embodiment, the tab member is releasable attached to the top layer, suitably by the use of an adhesive present on the tab member.

In another embodiment, the tab member is inseparably attached to the top layer, such as by welding.

In this context, the tab member may be releasable and/or inseparably attached to the top layer with the entire second surface thereof facing the top layer (i.e. the part of the tab member not releasable attached to the adhesive article), or with a smaller part thereof.

According to the invention, the tab member may also be partly inseparable attached to the top layer and partly releasable attached to the top layer.

According to a particular embodiment of the invention, said first portion of the tab member has a smaller area than said second portion of the tab member.

According to a preferred embodiment of the invention, the tab member and the top layer in combination with the bottom layer, is designed to be used as a grip for pulling apart the top layer and the bottom layer and exposing the adhesive surface of the adhesive article. Further, the tap member in combination with the top layer is designed to be used as a grip for applying the adhesive article without touching the adhesive surface of the article. In one embodiment, the tab member has a surface area sufficiently large to be handled by the use of the fingertips of one hand.

Suitably, the tab member has an area between 36 mm³ and 400 mm³, suitably between 36 mm³ and 400 mm³, and more suitably between 64 mm³ and 400 mm³.

Alternatively, the tab member may be formed by folding part of the top layer once upon itself to cover the second part of the adhesive surface of the adhesive article as well as a part of the top layer.

The tab member is a single layer optionally carrying a release layer, which is releasable attached to said second portion of said adhesive layer.

Suitable, the release layer on the tab member has an area corresponding to the area of said second end portion of the adhesive surface of the adhesive article or it has an area that is slightly larger than the area of the second end portion of the adhesive surface of the adhesive article. The rest of the surface of the tab member may optionally be attached to the surface of the top layer facing the tap member as describe above.

The release layer on the tab member is inseparably attached to the tab member.

Likewise, the release layer situated on the bottom layer is inseparably attached to the bottom layer.

Suitably the adhesive article is an adhesive wound dressing.

The invention also relates to a method for non-touch application of an adhesive article to a surface, where a layered product as described above is provided; the adhesive surface is exposed by gripping with one hand around the grip formed by the tap member and the top layer, and gripping with the other hand, the grip formed by the bottom layer, and pulling apart the top and bottom layer, while leaving the bottom layer and top layer attached to each other at the edge opposite the position of the tap member; applying the adhesive article to a surface using one hand and using the top layer as a dimensionally stable layer to support the adhesive article as it is placed on the surface, and separating the second portion of the adhesive article from the tap member from by pulling the grip formed by the tap member and the top layer along the surface to which the adhesive article has been applied in a direction away from the position of the adhesive article.

### DESCRIPTION OF THE PREFERRED EMBODIMENTS

The invention is now explained in more detail with reference to the drawings showing preferred embodiments of the invention.

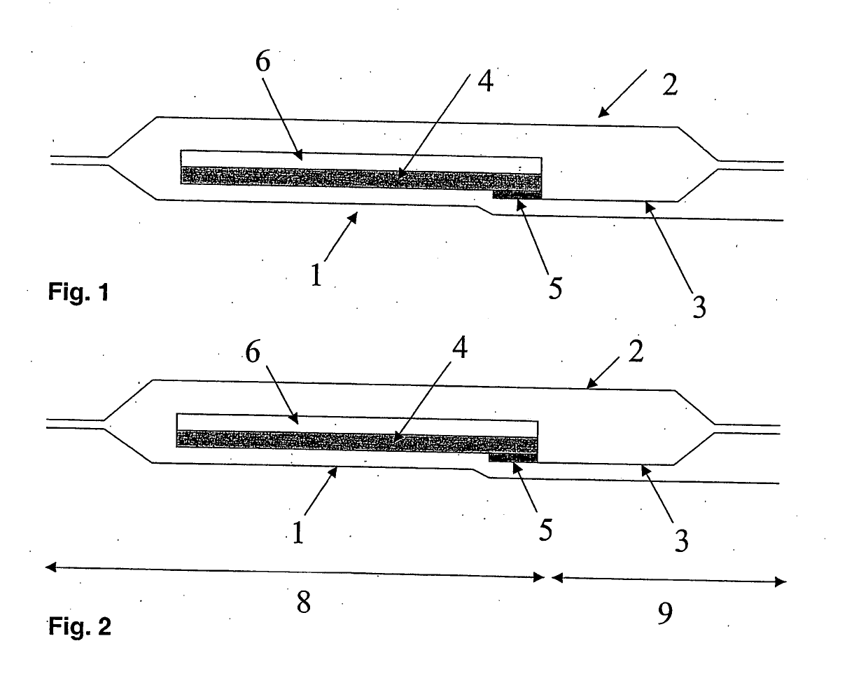

FIG. 1 is a longitudinal cross-sectional view of the layered product of the invention showing its essentially flat, planar layers. The figure illustrates the position of the bottom layer 1 which is releasable attached to the adhesive layer 4 and the top layer facing the cover layer 6 and the tap member 3. Also illustrated on the figure is the position of a portion of the adhesive layer 4 interposed between the top layer 2 and the tab member 3, and the adhesive surface of adhesive layer 4 facing the tab member 3, which is partly covered, by a release layer 5.

FIG. 2 is essentially the same figure as FIG. 1 but illustrates the embodiment of the invention where the top and bottom layer are sealed to each other forming a closed package. The portion 8 of the bottom layer is releasable attached and sealed to the top layer 2 and the tab member 3 and the portion 9 where the bottom layer is not attached to the tab member 3 nor the top layer, but where the tab member is releasably attached and/or sealed to the top layer, thereby forming a grip to be used for pulling apart the bottom layer 1 and the top layer 2.

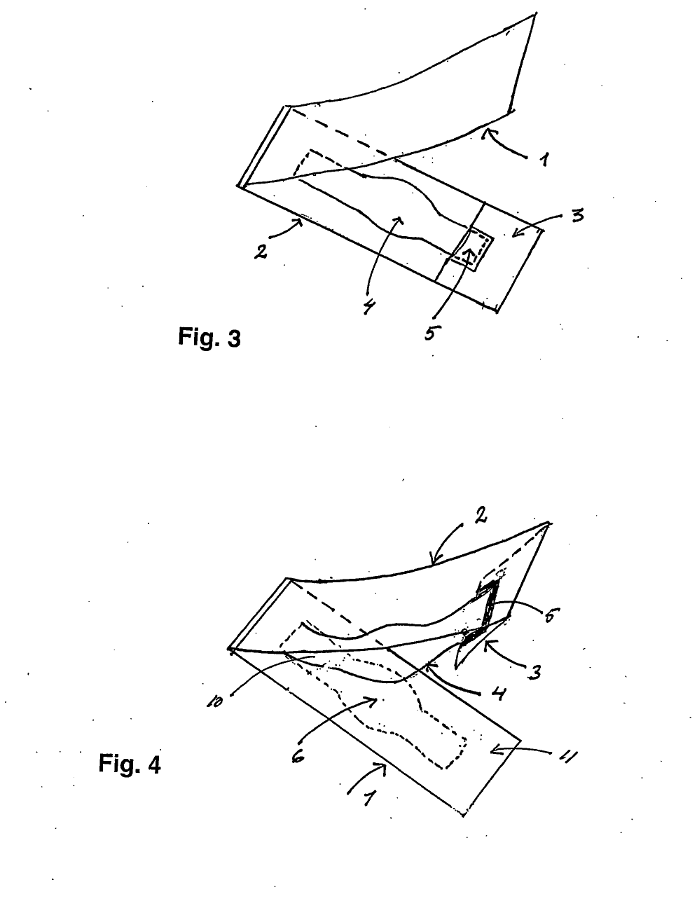

FIG. 3 illustrates the layered product of the invention after the top layer 2 and the bottom layer 1 has been pulled apart and the adhesive surface of the adhesive layer 4 has been exposed. The top layer 2 and bottom layer 1 are inseparably or releasable attached to each other at the edge opposite the position of the tab member. The figure illustrates the position of a release layer 5 on the tab member 3. The surface of the tab member 3 facing the surface of the top layer 2, may, or may not be attached to each other.

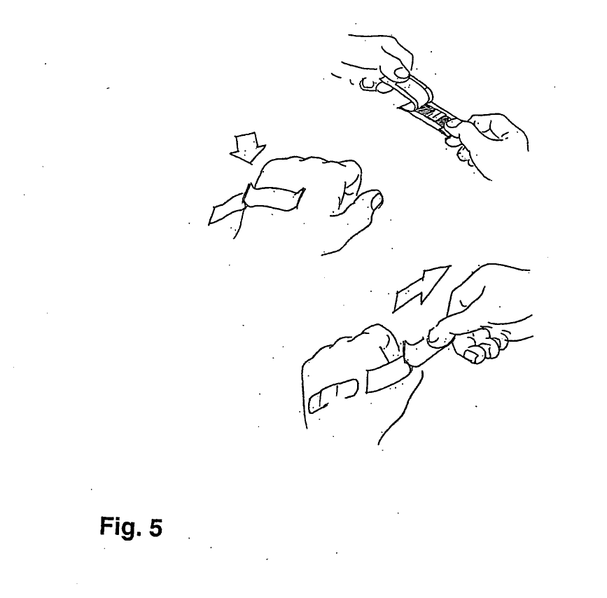

FIG. 4 also illustrates the layered product of the invention after the top layer 2 and the bottom layer 1 has been pulled apart and the adhesive surface of the adhesive layer 4 has been exposed. The top layer 2 and bottom layer 1 are inseparably or releasable attached to each other at the edge opposite the position of the tab member. The figure illustrates that the surface of the cover layer 10 is not attached to the top layer 2 but only to the tab member 3, which serves as a grip. The figure also illustrates an embodiment of the invention wherein the tab member 3 is only attached to the edge of the top layer 2. The figure illustrates the position of a release layer 5 on the tab member 3 and the area 6 illustrates the position of a possible release layer on the bottom layer 1 and 11 the position of a possible adhesive layer or cohesive layer on the bottom layer 1.

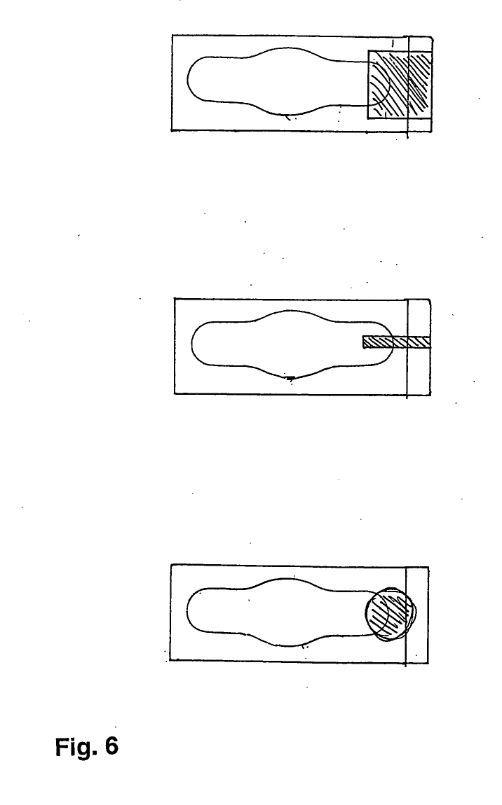

FIG. 5 illustrates how the top and bottom layers may be pulled apart and the adhesive article applied to the skin using only one hand and using the top layer as a dimensionally stable support during application of the adhesive layer to the skin.

FIG. 6 illustrates various shapes for the tab member. However, the figure may not be construed as limiting with respect to the shape of the tab member to the shapes shown.

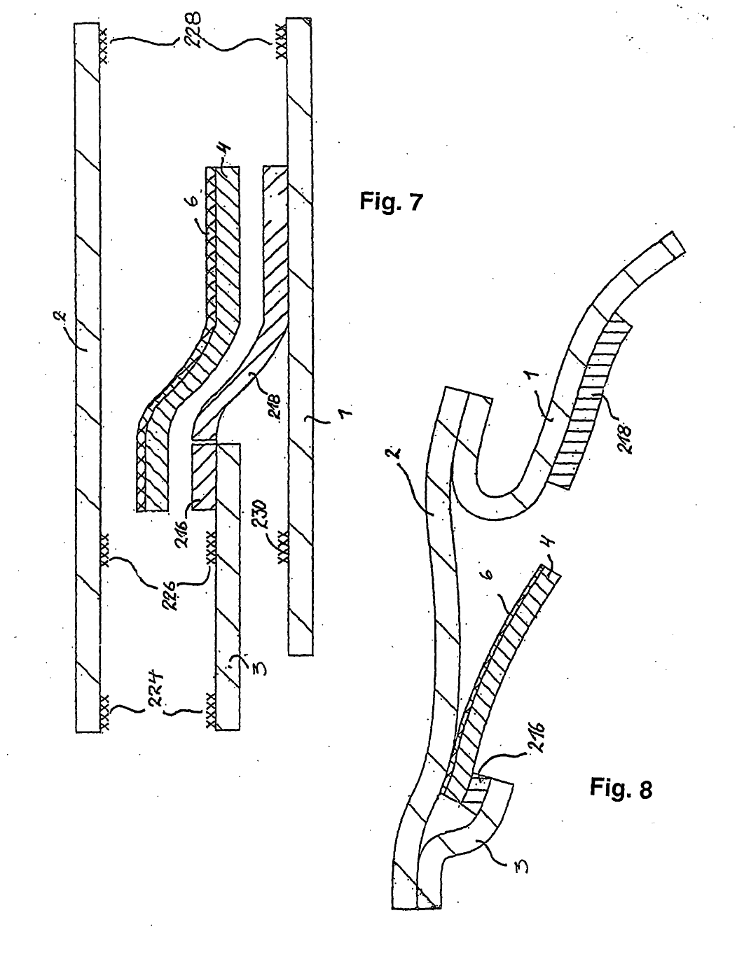

FIG. 7 illustrates the position of release layers and welding portions in an integrated package of the invention which comprises a top layer 2, first and second release layers 216 and 218, respectively, tab member 3 and bottom layer 1. Top layer 2 is secured to tap member 3 at welding portions 224 and 226 and to the bottom layer 1 at welding portion 228. The bottom layer 3 may be is secured to tab member 3 at welding portion 230.

FIG. 8 shows a configuration of the integrated package shown in FIG. 7 during application thereof. Bottom layer 1 has been separated from tab member 3 by braking welding 230, and release layer 218 has been peeled off adhesive layer 4. Cover layer 6 has been partially peeled off top layer 2, there being optionally provided an adhesive (not shown) for fixing the cover layer to the top layer 2. Once the adhesive 4 has been applied to the application site, release layer 216 is peeled off the adhesive. The release layers 216 and 218, which may be made from a continuous sheet of material, which is cut or punched to establish two distinct sheets, may e.g. be made from a silicone coated PETP sheet. The top layer 2, tab member 3 and bottom layer 1 may e.g. be made from foil of a plastics material.

Materials and Methods

The materials used for the various layers in the layered product of the invention may be any materials suitable for the particular use in the product. Some materials are mentioned below, but these materials should not be construed as limiting

Suitably the adhesive layer is an elastomeric pressure sensitive material optionally containing particles or polymers capable of absorbing water. Alternatively the adhesive layer may carry an absorbent pad of an absorbing material (gauze, foam or hydrocolloid), optionally carrying a pharmacologically active substance.

The adhesive layer may be selected from any number of known commercially available medical grade adhesives, such as acrylic, silicon, or polyisobutylene adhesives and may be mixed with various agents, such as drugs (antibiotics, antibacterial agents, pain killers) and water absorbing agents (hydrocolloids). A pharmacologically active substance may also be placed on the surface of the adhesive layer.

The cover layer may be made from any known material useful as a thin film on an adhesive dressing. Suitable materials include polyurethane, for example polyester or polyether polyurethanes, or elastomeric polyether polyesters film, polyether polyamides or hydrophilic polyurethanes.

The top and bottom layers and the tab member are suitably rectangular, ellipsoid or circular sheets although other shapes are also possible. Suitable, the top an/or bottom layer is made of siliconized paper or a plastic foil. Suitably, only a part of the surface of the bottom layer carry a silicone layer. Suitably the tab member, corresponds in shape and size to an end portion of the top layer.

The adhesive articles are suitable ellipsoid, circular or rectangular in shape although other shapes are also possible, see e.g. FIG. 6.

Adhesive layers for attaching the top layer to the bottom layer may be any conventional adhesive useful for this purpose.

Cohesive layers present on the top and bottom layer are typically selected from cohesive materials, which form a bond when activated, typically through pressure and heat. A suitable cohesive material is described in U.S. Pat. No. 2,529,060.

Other methods for attaching and/or sealing the top layer to the bottom layer are also covered by the present invention.

Methods for manufacturing the layered products of the invention are any suitable method in the art, preferably the methods described below.

Thus, the layered product of the invention, may be produced by a method involving
a) providing a top layer;
b) placing a cover layer of an adhesive article comprising the cover layer and an adhesive layer which two layers are inseparably fastened to each other, optionally in the form of discrete adhesive articles, on the top layer;
c) attaching the bottom layer having a release layer and a tab member on top of the bottom layer to the adhesive surface of the adhesive article;
d) die cutting the product into discrete portions still attached to the top layer,
e) optionally isolating the product by sealing the top layer to the bottom layer about the periphery of the adhesive article.

The layered product of the invention may be made on a conventional apparatus for preparation of such layered products. An advantage of the present invention is that the amount of top paper used during the process may be reduced because the same top paper is used from start to the end of the production. This is not normally convenient. The manufacturing process is also easier to synchronize.

1. A layered product for non-touch application of an adhesive article comprising a cover layer inseparably attached to an adhesive layer, said adhesive article being interposed between a top layer and a bottom layer where the top layer is situated proximate to the cover layer and the bottom layer situated proximate to the adhesive layer, said bottom layer having a release layer which is releasable attached to a first end portion of the adhesive surface of said adhesive layer characterized in that a second end portion of the adhesive surface of said adhesive layer is releasable attached to a first portion of a tap member which is attached to the top layer, or formed from a part of the top layer, and a second portion of said tab member forming a layer situated on the top layer.

2. A layered product according to claim 1 wherein the top layer and the bottom layer are fastened to each other, at least at the edge opposite the position of the tab member.

3. A layered product according to claim 1 wherein the bottom layer and the top layer have a surface area that is larger than the area of the adhesive article, and the top and bottom layer as well as the tab member and the top layer, are releasable sealed or attached to each other thereby forming a package containing the adhesive article.

4. A layered product according to claim 1 wherein the tab member is a separate member fastened to the top layer.

5. A layered product according to claim 1 wherein said first portion of the tab member has a smaller area than said second portion of the tab member.

6. A layered product according to claim 1 wherein the tab member and the top layer in combination with the bottom layer, is designed to be used as a non-touch grip for pulling apart the top layer and the bottom layer and exposing the adhesive surface of the adhesive article.

7. A layered product according to claim 1 wherein the tab member is a single layer optionally carrying a release layer, which is releasable attached to said second portion of said adhesive surface of the article.

8. A layered product according to claim 1, wherein an area of the bottom layer, situated peripherally to the release layer on the bottom layer is covered by an adhesive layer, which is releasable attached to the top layer.

9. A layered product according to claim 1 wherein a area of the top layer situated peripherally to the cover layer of the adhesive article, and optionally a part of the tab member, is covered by an adhesive layer which is releasable attached to the bottom layer.

10. A layered product according to claim 1 wherein an area of the bottom layer situated peripherally to the release layer on the bottom layer is covered by a cohesive layer and the surface of the top layer and tab member facing the bottom layer is covered by a cohesive layer essentially forming a mirror image of the cohesive layer on the bottom surface and said cohesive layers releasable attaching the top layer to the bottom layer.

11. A layered product according to claim 1 characterized in that more than 50% of the adhesive surface of the adhesive article is releasable attached to the release layer situated on the bottom layer and less than 50% of the adhesive surface area of the adhesive article is releasable attached to the tab member.

12. A layered product according to claim 11 characterized in that more than 85% of the adhesive surface of the adhesive article is releasable attached to the release layer situated on the bottom layer and less than 15% of the adhesive surface area of the adhesive article is releasable attached to the tab member.

13. A layered product according to claim 1 wherein the tab member has an area between 36 mm³ and 400 mm³, suitably between 64 mm³ and 400 mm³.

14. A layered product according to claim 1 wherein the tab member is formed by folding of the top layer once upon itself.

15. A layered product according to claim 1 wherein the tab member is a separate member fastened to the top layer at one or more edge(s) of the top layer or proximate to an edge(s) of the top layer.

16. A layered product according to claim 15 wherein the tab member is a separate member releasable attached to the top layer.

17. A layered product according to claim 15 wherein the tab member is a separate member inseparably attached to the top layer.

18. A layered product according to claim 1 wherein the tab member is releasable attached to the top layer over the entire surface of the tab member facing the top layer.

19. A layered product according to claim 1 wherein the tab member is inseparably attached to the top layer over the entire surface of the tab member facing the top layer.

20. A method for non-touch application of an adhesive article to a surface characterised in that a layered product according to claim 1 is provided; the adhesive surface is exposed by gripping with one hand around the grip formed by the tap member and the top layer, gripping with the other hand, the grip formed by the bottom layer and pulling apart the top and bottom layer, while leaving the bottom layer and top layer attached to each other at the edge opposite the position of the tap member; applying the adhesive article to the a surface using one hand only and using the top layer as a dimensionally stable layer to support the adhesive article as it is placed on the surface; and separating the second portion of the adhesive surface of the adhesive article from the tap member by pulling the grip formed by the tap member and the top layer along the surface to which the adhesive article has been applied.
